(19) United States
(12) Patent Application Publication
(10) Pub. No.: US 2022/0409793 A1
(43) Pub. Date: Dec. 29, 2022

(54) PERITONEAL DIALYSIS CASSETTE WITH EXTERNAL PUMP

(71) Applicant: Bellco, S.R.L, Mirandola (IT)

(72) Inventor: Gaspare Marotta, Mirandola (IT)

(73) Assignee: Bellco, S.R.L., Mirandola (IT)

(21) Appl. No.: 17/360,874

(22) Filed: Jun. 28, 2021

(51) Int. Cl. A61M 1/28

(52) U.S. Cl. CPC A61M 1/28; A61M 2205/3337; A61M 2205/3327; A61M 2205/3368; A61M 2205/3331; A61M 2205/50; A61M 2205/702

(57) ABSTRACT

A peritoneal dialysis cassette that uses an external pump to drive fluid through the peritoneal dialysis cassette is provided. The peritoneal dialysis cassette can include two inlet/outlet ports connected to an inlet and outlet of the external pump. The external pump drives fluid through fluid passages in the peritoneal dialysis cassette connecting a plurality of other inlet/outlet ports, with one or more valves usable to selectively direct fluid through the peritoneal dialysis cassette.

### FIELD

A peritoneal dialysis cassette that uses an external pump to drive fluid through the peritoneal dialysis cassette is provided. The peritoneal dialysis cassette can include two inlet/outlet ports connected to an inlet and outlet of the external pump. The external pump drives fluid through fluid passages in the peritoneal dialysis cassette connecting a plurality of other inlet/outlet ports, with one or more valves usable to selectively direct fluid through the peritoneal dialysis cassette.

### BACKGROUND

Conventional peritoneal dialysis systems often use a cassette to direct and control fluid movement for the generation of peritoneal dialysis fluid and delivery of peritoneal dialysis therapy to a patient. Often, the known systems use diaphragm pumps or other types of membrane-type pumps integrated within the cassette body. Such pumps and configurations require precise control over the pressure applied to membranes covering the diaphragm pumps during insertion into a cycler, priming for initial use, and actual use during therapy. The conventional cassettes typically require custom membranes useable with the diaphragm pumps and components to cause a vacuum wherein a diaphragm pump membrane is sucked flush against a pumping surface inside a cycler or similar device. The conventional systems and methods require a sealed airtight connection between a pumping surface on the cycler and a pumping surface on the cassette to engage and deliver a motive force. Such engagement between a movable diaphragm pump membrane on the cassette and pumping surface on the cycler generally requires pressure to induce and hold a vacuum such that when the pumping surface on the cycler retracts, the vacuum continues to hold the diaphragm pump membrane against the pumping surface to thus enlarge a chamber void left by the retracting diaphragm pump membrane, drawing fluid into one of the chambers. The conventional approaches require various pumps, sensors, and valves to maintain an airtight seal to provide the necessary vacuum and pressure to provide proper priming and continued functioning throughout an entire therapy session or however long the cassette is used. Such features and membrane requirements can be challenging and expensive.

As such, there is a need for systems, methods, and components that can use any type of external pump to move fluid through the peritoneal dialysis cassette. The need extends to cassettes and valves that can operate to selectively direct fluid through a cassette using an external rather than internal pump. The need extends to systems, methods, and components that do not rely on vacuum pressure to maintain an airtight seal. The need still further includes cassette systems for peritoneal dialysis that do not require the use of diaphragm pumps or other pumps requiring pressurized surfaces. The need includes systems and methods that do not require an airtight seal between a cassette surface and a surface of a component delivering a pumping action or motive force.

### SUMMARY OF THE INVENTION

The problem to be solved is the movement of fluid through a peritoneal dialysis cassette for any functions performed with a peritoneal dialysis system without the need for pumps or pumping chambers within the peritoneal dialysis cassette. The solution is to include two inlet/outlet ports fluidly connectable to an external pump to provide the motive force for moving fluid through the peritoneal dialysis cassette.

The first aspect relates to a peritoneal dialysis cassette. In any embodiment, the peritoneal dialysis cassette can include a cassette housing having a plurality of fluid channels fluidly connected to a plurality of inlet/outlet ports; wherein the plurality of inlet/outlet ports has at least a first inlet port fluidly connectable to an external pump and a second inlet/outlet port fluidly connectable to the external pump; and one or more valve positions in the plurality of fluid channels to selectively direct fluid from a first specified inlet/outlet port to a second specified inlet/outlet port; the external pump fluidly connectable to the cassette housing via the plurality of inlet/outlet ports.

In any embodiment, the peritoneal dialysis cassette can include a conductivity sensor in a fluid line fluidly connecting the external pump to either the first or second inlet/outlet port.

In any embodiment, the peritoneal dialysis cassette can include an air bubble detector in a fluid line fluidly connecting the external pump to either the first or second inlet/outlet port.

In any embodiment, the peritoneal dialysis cassette can include at least one contactless temperature sensor in the peritoneal dialysis cassette.

In any embodiment, the peritoneal dialysis cassette can include at least one pressure sensor in the peritoneal dialysis cassette.

The features disclosed as being part of the first aspect can be in the first aspect, either alone or in combination, or follow any arrangement or permutation of any one or more of the described elements. Similarly, any features disclosed as being part of the first aspect can be in a second or third aspect described below, either alone or in combination, or follow any arrangement or permutation of any one or more of the described elements.

The second aspect relates to a system. In any embodiment, the system can include the peritoneal dialysis cassette of the first aspect; a first peritoneal dialysis fluid source fluidly connectable to a third inlet/outlet port; a peritoneal dialysis fluid bag fluidly connectable to a fourth inlet/outlet port; and a control system in communication with one or more valves each positioned at one of the one or more valve positions; the control system programmed to control the one or more valves to selectively direct fluid through the peritoneal dialysis cassette.

In any embodiment, the system can include at least a second peritoneal dialysis fluid source fluidly connectable to a fifth inlet/outlet port; the control system programmed to control the one or more valves to selectively direct fluid from the first peritoneal dialysis fluid source to the peritoneal dialysis fluid bag; and then to selectively direct fluid from the second peritoneal dialysis fluid source to the peritoneal dialysis fluid bag.

In any embodiment, the system can include a fifth inlet/outlet port fluidly connectable to a patient line; the control system programmed to control the one or more valves to selectively direct fluid from the peritoneal dialysis fluid bag into the patient line.

In any embodiment, the system can include a fifth inlet/outlet port fluidly connectable to a drain line; the control system programmed to control the one or more valves to selectively direct fluid from the peritoneal dialysis fluid bag into the drain line.

In any embodiment, the system can include a sixth inlet/outlet port fluidly connectable to a drain line; the control system programmed to control the one or more valves to selectively direct fluid from the patient line into the drain line.

In any embodiment, the system can include a fifth inlet/outlet port and a sixth inlet/outlet port fluidly connected to a sampling line.

In any embodiment, the sampling line can be fluidly connected to a sampling bag.

In any embodiment, the system can include a conductivity sensor in a fluid line fluidly connecting the external pump to either the first or second inlet/outlet port.

In any embodiment, the system can include a sixth inlet/outlet port fluidly connectable to a patient line; wherein the control system is programmed to control the one or more valves to selectively direct fluid from the fourth inlet/outlet port to the sixth inlet/outlet port.

In any embodiment, the system can include an air bubble detector in a fluid line connecting the external pump to either the first or second inlet/outlet port.

In any embodiment, the system can include a sixth inlet/outlet port fluidly connectable to a patient line; wherein the control system is programmed to control the one or more valves to selectively direct fluid from the fourth inlet/outlet port to the sixth inlet/outlet port.

The features disclosed as being part of the second aspect can be in the second aspect, either alone or in combination, or follow any arrangement or permutation of any one or more of the described elements. Similarly, any features disclosed as being part of the second aspect can be in the first or third aspect, either alone or in combination, or follow any arrangement or permutation of any one or more of the described elements.

The third aspect relates to a method using the peritoneal dialysis cassette of the first aspect. In any embodiment, the method can include the steps of pumping fluid from a third inlet/outlet port fluidly connected to a peritoneal dialysis fluid source, through the peritoneal dialysis cassette to a fourth inlet/outlet port fluidly connected to a peritoneal dialysis fluid bag.

In any embodiment, the method can include the step of pumping fluid from the peritoneal dialysis fluid bag into a patient line fluidly connected to a fifth inlet/outlet port; the patient line fluidly connectable to a catheter.

In any embodiment, the method can include the step of measuring at least one fluid characteristic of fluid in a fluid line fluidly connecting the external pump to either the first or second inlet/outlet port prior to pumping the fluid to the patient line.

In any embodiment, the fluid line fluidly connecting the external pump to either the first or second inlet/outlet port can include at least a conductivity with temperature sensor and an air bubble detector.

The features disclosed as being part of the third aspect can be in the third aspect, either alone or in combination, or follow any arrangement or permutation of any one or more of the described elements. Similarly, any features disclosed as being part of the third aspect can be in the first or second aspect, either alone or in combination, or follow any arrangement or permutation of any one or more of the described elements.

### BRIEF DESCRIPTION OF THE DRAWINGS

FIGS. 1A-D illustrate a peritoneal dialysis cassette using an external rotary or vane pump.

FIGS. 2A-B illustrate a linear actuator with rigid shaft and soft head for use with the peritoneal dialysis cassette.

FIG. 3A illustrates the peritoneal dialysis cassette assembly with sensors and linear actuator footprint in performing peritoneal dialysis therapy.

FIGS. 3B-F illustrate the use of the peritoneal dialysis cassette in performing peritoneal dialysis therapy.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

An “air bubble detector” is a device capable of detecting the presence of one or more bubbles in a liquid.

The term “cassette housing” refers to the outer rigid walls of a cassette.

The term “communication” refers to electronic communication of any type.

The term “comprising” includes, but is not limited to, whatever follows the word “comprising.” Use of the term indicates the listed elements are required or mandatory but that other elements are optional and may be present.

A “conductivity sensor” is device for measuring the electrical conductance of a solution and/or the ion, such as a sodium ion, concentration of a solution.

A “temperature sensor” is device for measuring the temperature of a solution with or without contact.

The term “connected” or to “connect” refers to physical contact between two or more components.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

A “control system” can be a combination of components that act together to maintain a system to a desired set of performance specifications. The control system can use processors, memory and computer components configured to interoperate to maintain the desired performance specifications. The control system can receive data from sensors to determine a state of a system and control one or more components to maintain a desired performance specification. The control system can also include fluid or gas control components, and solute control components as known within the art to maintain performance specifications.

A “drain line” is a fluid line through which used or waste fluid can be disposed.

The term “external pump” refers to a pump that is positioned outside of a cassette.

A “fluid channel” is a conduit through which a fluid can move.

A “fluid characteristic” is any parameter of a fluid, including concentration of one or more solutes, temperature, fluid pressure, or any other parameter.

The term “fluidly connectable” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

The term “fluid line” refers to any tubing or conduit through which fluid can travel.

An “inlet/outlet port” is an opening or conduit through which fluids can enter or exit a component.

The term “measuring” or to “measure” refers to determining a characteristic of a substance, fluid, or system.

A “patient line” refers to a fluid line fluidly connectable to a catheter for infusion of fluid into a patient or removal of fluid from a patient.

A “peritoneal dialysis cassette” is a housing containing one or more fluid channels that can be connected to fluid lines or components of a peritoneal dialysis system.

“Peritoneal dialysis fluid” is a dialysis solution to be used in peritoneal dialysis having specified parameters for purity and sterility. Peritoneal dialysis fluid is not the same as dialysate fluid of the type used in hemodialysis.

A “peritoneal dialysis fluid source” is a container of any type that holds components used to generate peritoneal dialysis fluid.

A “peritoneal dialysis fluid bag” is a container of any type that holds peritoneal dialysis fluid prior to infusion of the peritoneal dialysis fluid into a patient.

A “pinch valve” is a device capable of directing the flow of fluid or gas by pushing on a flexible portion of a fluid pathway, obstructing one or more pathways to allow the fluid or gas to travel in a path.

A “pinch valve position” is a placement in a fluid channel at which a pinch valve is placed to selectively direct fluid through the fluid channel. In certain embodiments, a valve position can be a valve. Alternatively, the valve position can be a portion of a fluid channel with which a valve can interact to selectively direct the fluid.

A “pressure sensor” is a component or set of components capable of determining a force exerted by a fluid in a system.

The term “programmed,” when referring to a processor or control system, can mean a series of instructions that cause a processor or control system to perform certain steps.

The term “pump” refers to any device that causes the movement of fluids or gases by applying suction or positive pressure.

The term “selectively directing” or to “selectively direct” refer to causing fluid to move through a system in a specified pathway.

A “sampling bag” is a container into which fluid to be analyzed can be collected.

A “sampling line” is a fluid line through which fluid having at least one fluid characteristic to be measured is flowed. The sampling line can include at least one sensor to measure at least one fluid characteristic of fluid in the fluid line, or can be connected to a bag or container for later analysis of the fluid.

A “temperature sensor” is a device capable of determining the temperature of a substance, surface, or fluid.

### Dialysis Cassette

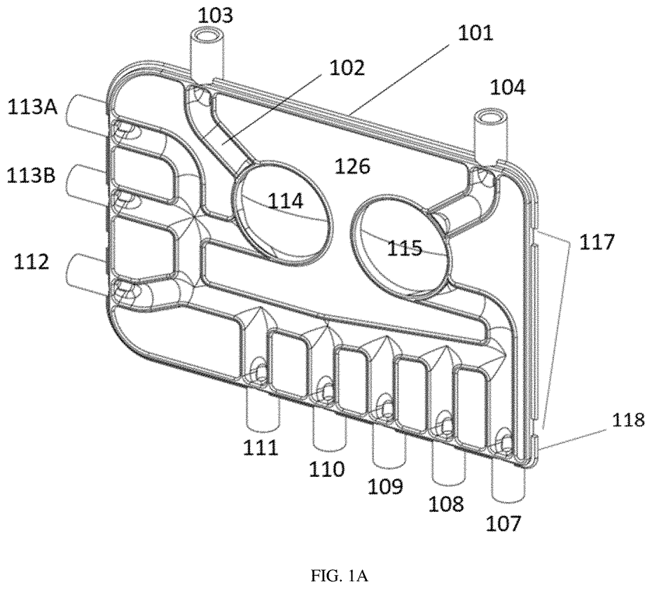

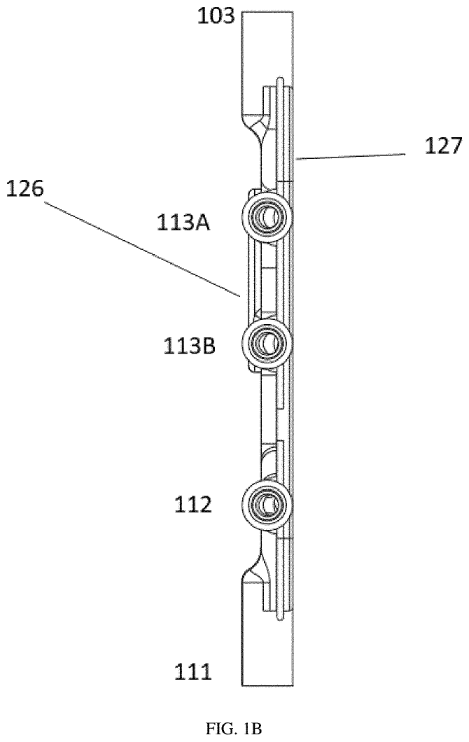

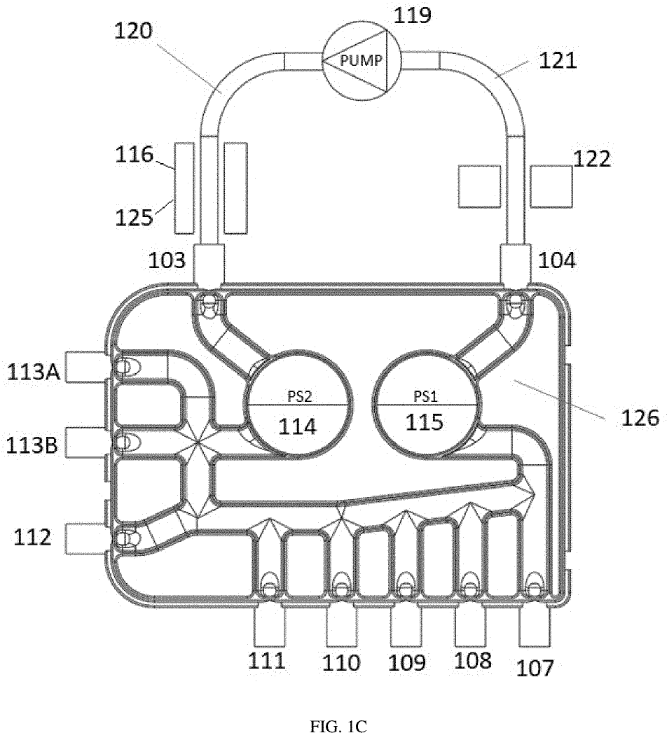

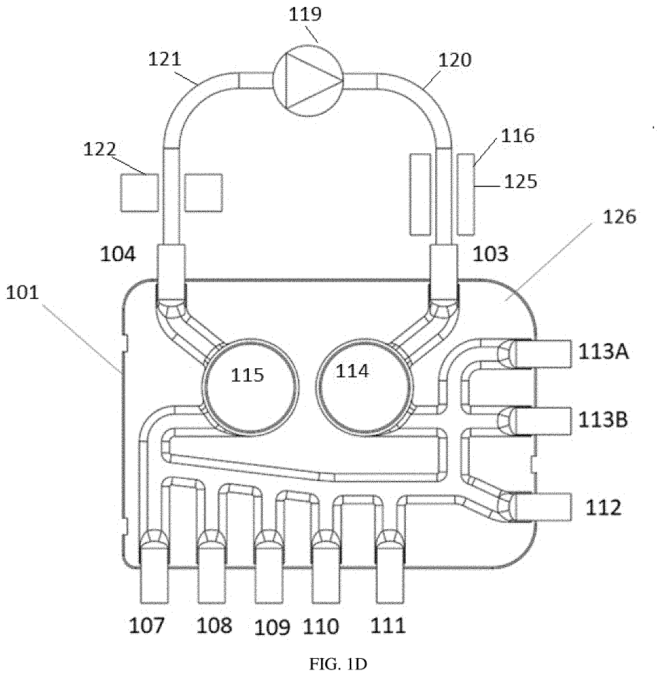

FIGS. 1A-D illustrate a peritoneal dialysis cassette. FIG. 1A is a perspective view of the peritoneal dialysis cassette, FIG. 1B is a side view of the peritoneal dialysis cassette, FIG. 1C is a front view of the peritoneal dialysis cassette connected to an external pump 119 and FIG. 1D is a back side of the peritoneal dialysis cassette. In FIG. 1A, the peritoneal dialysis cassette can include a cassette housing 101 enclosing a plurality of fluid channels 102. The cassette housing 101 can have a first surface that is a rigid surface 126. The fluid channels 102 can be cut into the rigid surface 126 or fabricated by any suitable manufacturing process such as a thermoplastic molding. The cassette housing 101 can also have a flexible membrane surface (not visible in FIG. 1A). One or more pinch valves (not shown) can press on the membrane surface, pushing the membrane to the rigid surface 126 of the cassette housing 101, and occluding a portion of the fluid channels 102. A control system (not shown) in communication with the one or more valves can be programmed to operate the valves to selectively direct fluid through the peritoneal dialysis cassette.

As described, the system can use pinch valves to control the movement of fluid through the peritoneal dialysis cassette fluid paths. Pinch valves can include a rigid shaft with a soft head; the deformation function of the soft head, allows to perfectly follow the cross-section profile of the fluid path for a complete occlusion. When the pinch valve extends, the soft head can press on the flexible membrane surface, forcing the membrane into the fluid channel 102 obstructing a fluid pathway. The control system can control the pinch valves in any manner known in the art. In certain embodiments, the system can include a pneumatic system for controlling the pinch valves movement. The control system can operate one or more pneumatic valves to pump air into or out of the pinch valves, causing the pinch valves to extend and obstruct a portion of the fluid channels 102. In certain embodiments, the system can use an electromechanical system for controlling the pinch valves. The control system can operate one or more motors in communication with the pinch valves to extend or retract the soft head of the pinch valve, thereby controlling fluid movement through the fluid channels 102. Alternatively, the system can use a hybrid pneumatic and electromechanical system for controlling the pinch valves. The control system can operate one or more combination of motors and pneumatic system in communication with the pinch valves to extend or retract the soft head of the pinch valve, thereby controlling fluid movement through the fluid channels 102.

The fluid channels 102 connect inlet/outlet ports of the peritoneal dialysis cassette. An external pump (not shown in FIG. 1A, but shown in FIGS. 1C-D) can be fluidly connected to inlet/outlet port 103 and inlet/outlet port 104. Operating the external pump in a will direct fluid from inlet/outlet port 103 to inlet outlet port 104. In certain embodiments, the external pump can be a bidirectional pump that can also direct fluid from inlet/outlet port 104 to inlet/outlet port 103.

In certain embodiments, the peritoneal dialysis cassette can include one or more sensors to measure fluid characteristics within the peritoneal dialysis cassette. For example, position 114 and position 115 of the peritoneal dialysis cassette can be used for a pressure sensor. A sensor can contact the membrane surface of the peritoneal dialysis cassette and measure the force exerted by fluid inside the fluid channels 102 at position 114 and position 115. Other sensors, such as a contactless temperature sensor (not shown in FIG. 1A) can be included to measure the temperature of fluid within the fluid channels 102.

As described, the fluid channels 102 connect various input/output ports. For example, in FIGS. 1A-D, the fluid channels 102 provide for fluid connections between inlet/outlet port 107, inlet/outlet port 108, inlet/outlet port 109, inlet/outlet port 110, inlet/outlet port 111, inlet/outlet port 112, inlet/outlet port 113A, and inlet/outlet port 113B. Inlet/outlet port 113A and inlet/outlet port 113B can be used as a draining system with two precise functions: (a) inlet/outlet port 113A can be a standard drain connector for continuous waste fluid expulsion during the entire dialysis treatment; (b) inlet/outlet port 113B can be a connector for waste fluid collection into a sterile sampling bag or sensor line (not shown). Various sensors, such as conductivity sensors, osmotic agent sensors, or any other sensor can be included to measure a fluid parameter of fluid in a sensor line connected to inlet/outlet port 113B or in fluid directed to a sampling bag fluidly connected to inlet/outlet port 113B. The inlet/outlet ports can be connected to various components of a peritoneal dialysis system, allowing control over the movement of fluid between the various components for generation of peritoneal dialysis fluid, infusion of the peritoneal dialysis fluid into a patient, drainage of used fluid from the patient, and/or cleaning and disinfection of the system. Although illustrated with eight inlet/outlet ports in addition to the inlet/outlet ports connecting to the pump and sensor line, one of skill in the art will understand that the peritoneal dialysis cassette can include any number of inlet/outlet ports depending on the number of components connected to the peritoneal dialysis cassette. Similarly, any number of valves can be included to direct fluid from any inlet/outlet port to any other inlet/outlet port.

FIG. 1B is a side view of the peritoneal dialysis cassette. As described, the cassette housing 101 can include a rigid surface (or entire body) 126 and a flexible membrane surface 127. The cassette housing 101 can be constructed to have a relatively slim side profile and can be engineered to be inserted into a slot or receiving compartment of a dialysis system. Fluid lines can connect inlet/outlet port 103 to a pump (not shown in FIG. 1B) to drive fluid through the peritoneal dialysis cassette. A drain line (not shown in FIG. 1B) can connect inlet/outlet port 113A and a sampling line can connect to inlet/outlet port 113B. Fluid lines fluidly connected to various components of a peritoneal dialysis system can be fluidly connected to inlet/outlet port 112, as well as the other inlet/outlet ports (not shown in FIG. 1B).

FIG. 1C illustrates the peritoneal dialysis cassette connected to an external pump 119. The external pump 119 can connect to the peritoneal dialysis cassette housing 101 through a plurality of inlet/outlet ports. As illustrated in FIG. 1C, the external pump 119 can connect to the peritoneal dialysis cassette housing 101 through external fluid line 120 connected to inlet/outlet port 103 and external fluid line 121 connected to inlet/outlet port 104. By pumping fluid from inlet/outlet port 103 to inlet/outlet port 104, or vice versa, fluid can be flowed through the fluid channels 102 from a first specified inlet/outlet port to a second specified inlet/outlet port. As illustrated in FIG. 1C, the external pump 119 can be a volumetric pump, as well as rotary pump. However, because the pump is external to the peritoneal dialysis cassette housing 101, the peritoneal dialysis cassette can be connected to any type of external pump. Further, because the external pump 119 is not inside the peritoneal dialysis cassette housing 101, the peritoneal dialysis cassette can be smaller, simpler, and cheaper to manufacture.

As described, the peritoneal dialysis cassette can include sensors using external fluid lines 120 and 121. Fluid pumped through external pump 119 via inlet/outlet port 103 and inlet/outlet port 104 can travel through fluid line 120 and fluid line 121. The fluid line 120 can include a first sensor 116 and a second sensor 125. The first sensor 116 and a second sensor 125 can be positioned on opposing sides of the fluid line 120 as shown in FIG. 1C. Alternatively, the first sensor 116 and a second sensor 125 can be positioned in any suitable position with respect to fluid line 120 known to one of skill in the art to improve performance or detection ability of the sensor. Additional sensors can be added as required. The sensors can include a temperature sensor, conductivity sensor, and combinations thereof. Fluid line 121 can include an air bubble detector 122. Alternative or additional sensors can be included, such as additional pressure sensors, temperature sensors, refractive index sensors, ion selective electrodes, air bubble detectors, or any other type of sensor. Although shown as positioned to measure fluid characteristics in fluid line 120 and fluid line 121, the first sensor 116, air bubble sensor 122, and the second sensor 125 can alternatively be included in a separate sampling line (not shown) fluidly connected to additional inlet/outlet ports. The control system can be in communication with any one or more of the described sensors to control the process. For example, if the composition of peritoneal dialysis fluid does not match the patient prescription, the control system can control one or more valves to direct fluid to a drain line rather than a patient line while providing a message or alert to the user. If the composition of the peritoneal dialysis fluid does match the patient prescription, the control system can direct the fluid into the patient line for infusion into a patient.

Using an external pump 119, rather than a diaphragm pump within the peritoneal dialysis cassette avoids the need for dedicated space for a pump. As such, a peritoneal dialysis cassette can be smaller and simpler. Using an external pump 119 can also free up additional space within the peritoneal dialysis cassette for fluid channels, sensors, and valves. Peritoneal dialysis cassettes that use diaphragm pumps require a vacuum seal to accurately control the diaphragm pump. However, because the external pump 119 can be any type of fluid pump, precise control over pressurization and creation of a vacuum seal of the peritoneal dialysis cassette to a base device or console is not required to mate a pumping surface on the base device for accurate delivery of fluid through the peritoneal dialysis cassette. The peritoneal dialysis cassette can simply be placed into a housing of the peritoneal dialysis system and connected to fluid lines at the inlet/outlet ports. In certain embodiments, the external pump 119 can be a disposable pump that is replaced after each use, or after a set number of uses. Alternatively, the external pump 119 can be a reusable pump that is cleaned and disinfected along with the rest of the peritoneal dialysis system.

FIG. 1D illustrates the opposite side of the peritoneal dialysis cassette housing 101 can include the back side of rigid surface 126 which interacts with the mechanical part of the control unit (not shown).

In certain embodiments, sensors can be used to measure fluid parameters while within the cassette housing 101. For example, position 114 and position 115 of the peritoneal dialysis cassette can be used for a pressure sensor that measures the fluid pressure on the membrane surface of the cassette housing 101.

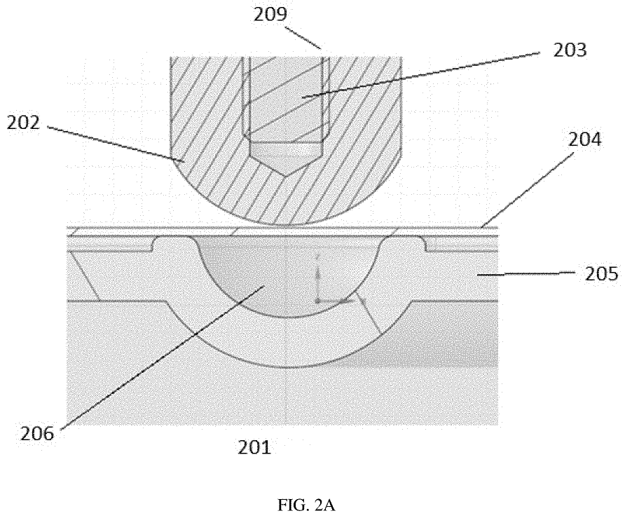

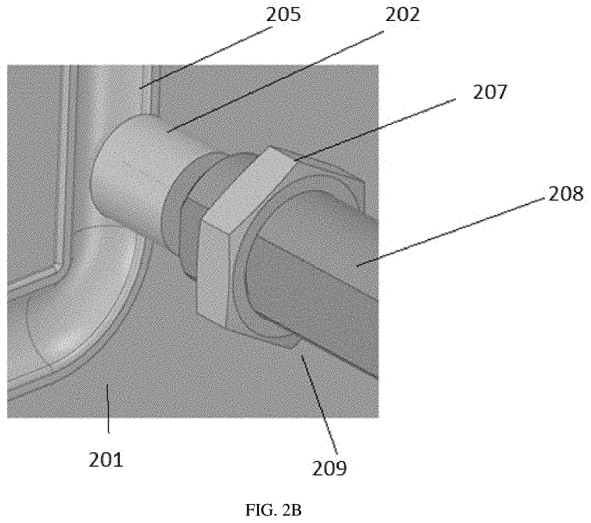

FIGS. 2A 2B illustrate a linear actuator that can be used as a pinch valve to selectively direct fluid through the peritoneal dialysis cassette. FIG. 2A is a cross-sectional view of a linear actuator 209 and peritoneal dialysis cassette 201. FIG. 2B is a perspective view of the linear actuator 209 and peritoneal dialysis cassette 201.

As described, and illustrated in FIG. 2A, the peritoneal dialysis cassette 201 can include a first membrane surface 204 and a second rigid surface 205. The area between the membrane surface 204 and rigid surface 205 defines a fluid channel 206 through the peritoneal dialysis cassette. One end of the linear actuator 209 can include a rigid central shaft 203 covered by a pinch bud 202. Any suitable material can be used for the pinch bud 202 including a plastic, rubber, thermoplastic, or combinations or mixtures thereof. The pinch bud 202 can be at any suitable hardness. For example, the pinch bud 202 can be made of a soft material to prevent damages to the first membrane surface 204. The linear actuator 209 can be positioned on the membrane surface 204 side of the peritoneal dialysis cassette 201. In certain embodiments, when air is directed into the linear actuator with a pneumatic system (not shown) the linear actuator extends, pushing the pinch bud 202 into the membrane surface 204 of the peritoneal dialysis cassette 201. The membrane surface 204 is pushed to the rigid surface 205 of the peritoneal dialysis cassette 201, occluding and preventing fluid movement through the fluid channel 206. When air is vented out of the linear actuator 209, the linear actuator 209 retracts, allowing the membrane surface 204 to move away from the rigid surface 205, and allowing fluid movement through the fluid channel 206. In certain embodiment, when the electromechanical system is activated, the linear actuator extends using an internal motor, pushing the pinch bud 202 into the membrane surface 204 of the peritoneal dialysis cassette 201. The membrane surface 204 is pushed to the rigid surface 205 of the peritoneal dialysis cassette 201, occluding and preventing fluid movement through the fluid channel 206. When the electromechanical system is de-activated, the linear actuator 209 retracts reversing the motor direction, allowing the membrane surface 204 to move away from the rigid surface 205, and allowing fluid movement through the fluid channel 206. Alternatively, in certain embodiments, a combination of electromechanical- and pneumatic system allows to the linear actuator 209 to extend and retract creating the occlusion allowing fluid movement or not through the fluid channel 206.

As illustrated in FIG. 2B, the linear actuator 209 can have an outer shaft 208, with the extendible rigid shaft 203 (not shown in FIG. 2B) inside the outer shaft 208. The pinch bud 202 is positioned over a fluid channel of the peritoneal dialysis cassette 201, such that when extended, the pinch bud 202 pushes the membrane surface 204 to the rigid surface 205 to occlude the fluid channel. Nut 307 can be used to couple the linear actuator 209 to an internal surface of a control unit (not shown) that controls the linear actuator 209.

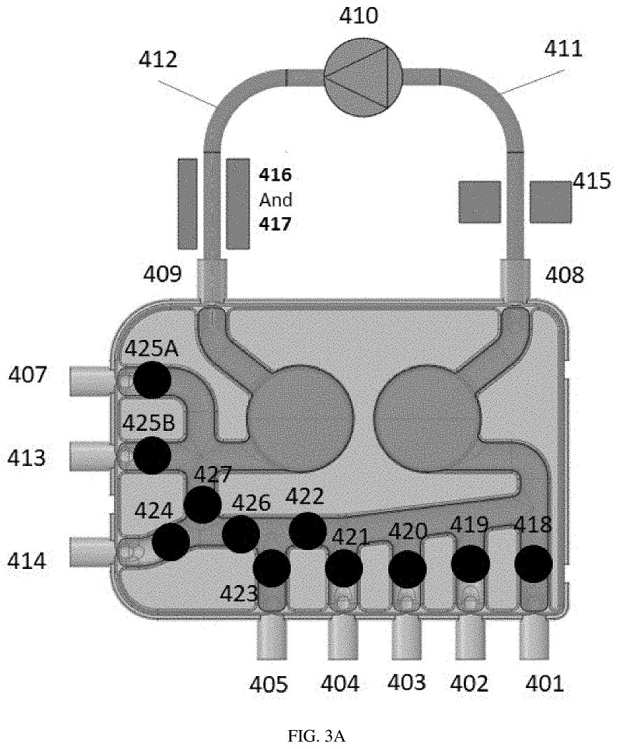

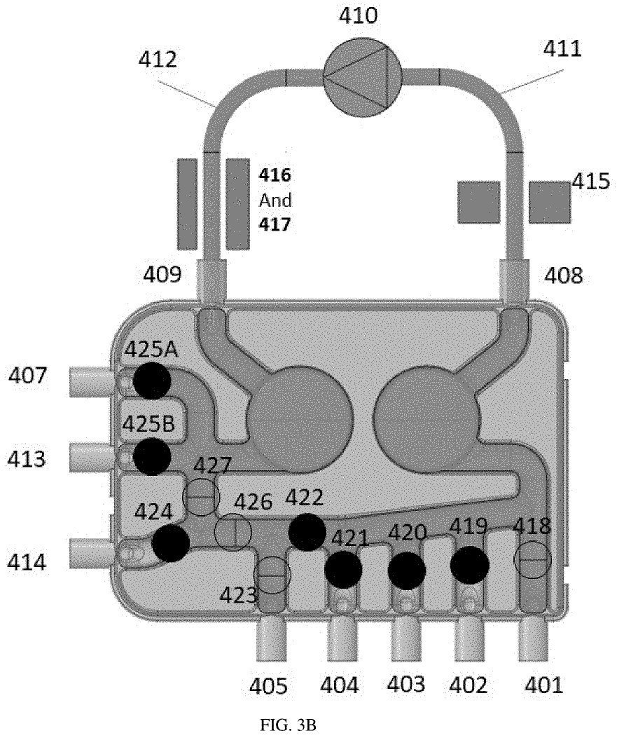

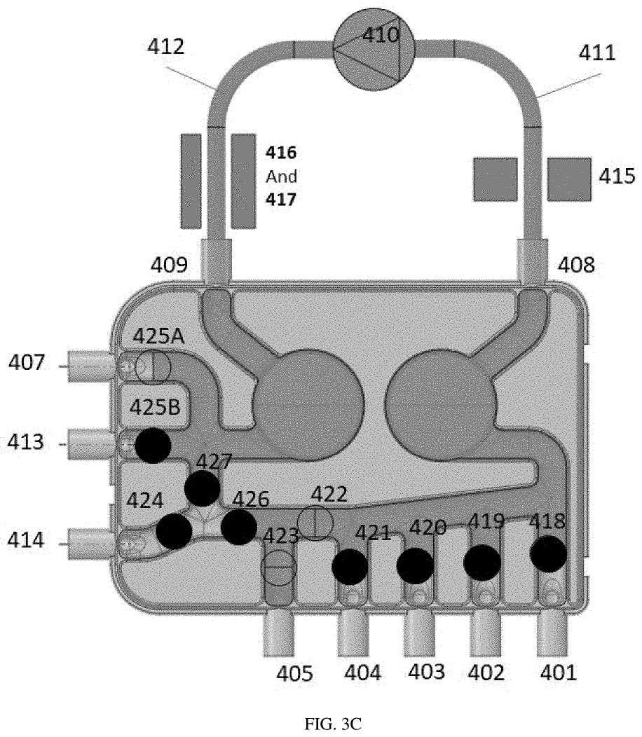

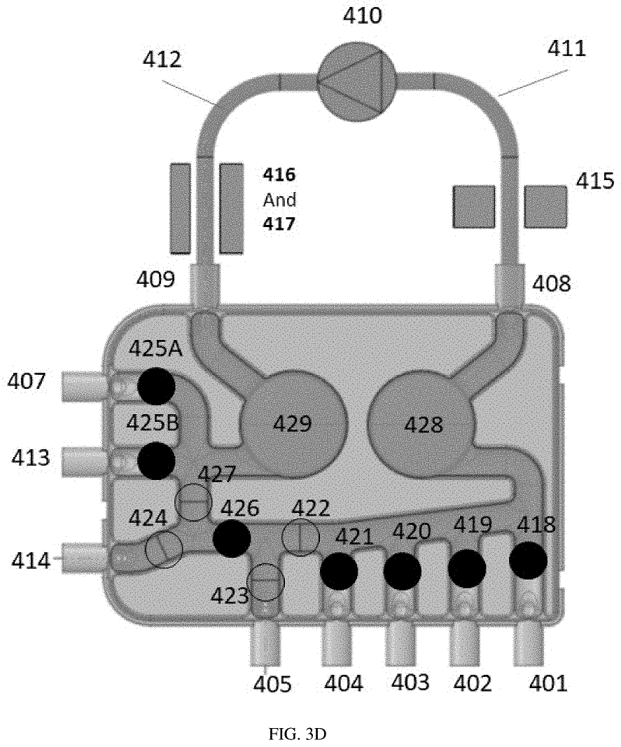

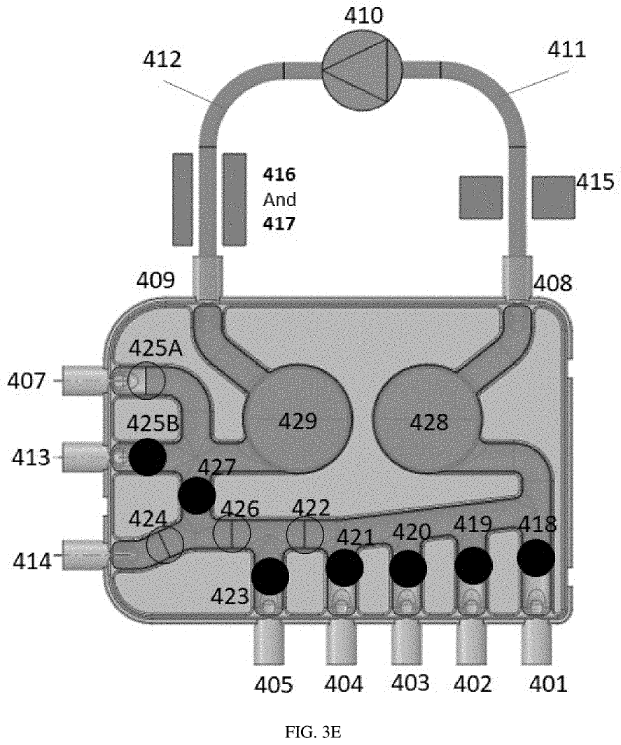

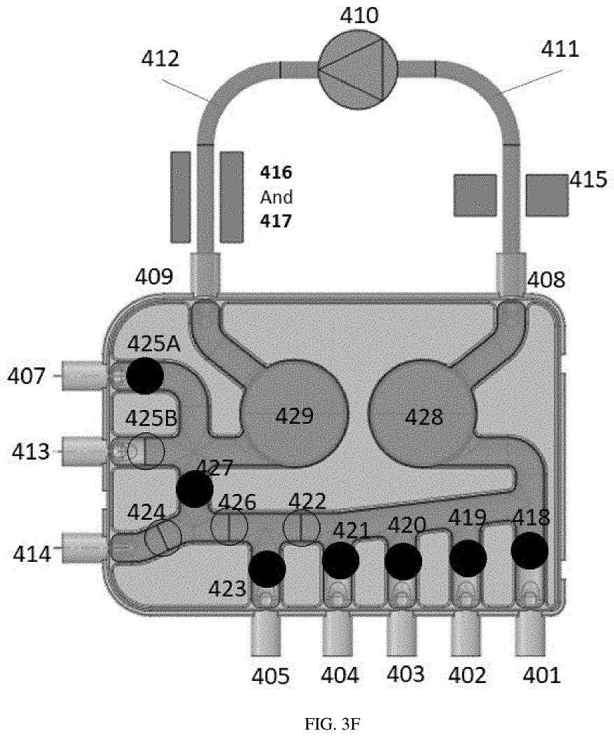

FIGS. 3A-E illustrate the use of a peritoneal dialysis cassette. FIG. 3A is the initial resting state of the peritoneal dialysis cassette, FIG. 3B shows the movement of fluid for generating peritoneal dialysis fluid from one or more constituent components, FIG. 3C shows the movement of fluid for priming the peritoneal dialysis cassette with peritoneal dialysis fluid, FIG. 3D shows the movement of fluid for filling a patient, FIG. 3E shows the movement of fluid for draining used peritoneal dialysis fluid from the patient, and FIG. 3F shows the movement of fluid for waste fluid collection. In each of FIGS. 3A-F, closed valves, occluding fluid channels, are shown as dark circles, while open valves that allow fluid to pass through the fluid channels are shown as open circles.

As described, the peritoneal dialysis cassette can include inlet/outlet ports for connections between several components. In each of FIGS. 3A-F, inlet/outlet port 401 is fluidly connectable to the first pre-filled bag; inlet/outlet port 402 is fluidly connectable to a second pre-filled bag; inlet/outlet port 403 is fluidly connectable to a third pre-filled bag; inlet/outlet port 404 is fluidly connectable to a fourth pre-filled bag; inlet/outlet port 407 is fluidly connectable to a drain line, inlet/outlet port 413 is fluidly connected to a sampling bag or a sampling line, and inlet/outlet port 405 is fluidly connectable to a peritoneal dialysis fluid bag; inlet/outlet port 414 is fluidly connectable to a patient line. However, one of skill in the art will understand that the components each inlet/outlet port are connected to are provided for illustrative purposes only. Additional or fewer components can be connected through the peritoneal dialysis cassette and the location of each inlet/outlet port connected to each component can be changed.

In FIGS. 3A-F, the system uses four pre-filled bags as a peritoneal dialysis fluid source containing concentrated solutions of various peritoneal dialysis components. Solutions from each of the pre-filled bags are added to the peritoneal dialysis fluid bag for mixing and heating and then infused into the patient. However, the described peritoneal dialysis cassette can work with additional or fewer pre-filled bags, or can work with a single peritoneal dialysis fluid bag already having all of the components of the final peritoneal dialysis fluid.

FIG. 3A shows the peritoneal dialysis cassette in the initial state. Linear actuators or any other type of pinch valve are positioned on the membrane side of the peritoneal dialysis cassette to serve as valves. The peritoneal dialysis cassette illustrated in FIGS. 3A-F use 11 linear actuators serving as valve 418, valve 419, valve 420, valve 421, valve 422, valve 423, valve 424, valve 425A, valve 425B, valve 426, and valve 427. However, the peritoneal dialysis cassette can be arranged with more or fewer valves, depending on the needs of the system. In certain embodiments, rotary valves or other types of valves can be used in place of the linear actuators. As described, the peritoneal dialysis cassette can include position 428 and position 429 for pressure sensors. Additional sensors can be included in fluid line 411 and fluid line 412, such as air bubble detector 415, temperature sensor 417, and conductivity sensor 416.

FIG. 3B illustrates the use of the peritoneal dialysis cassette in filling a peritoneal dialysis fluid bag from one or more peritoneal dialysis fluid sources. Opening valve 418, valve 427, valve 426, and valve 423 allows a concentrate from a first concentrate source to be pumped from inlet/outlet port 401 to inlet outlet port 408 and into fluid line 411. External pump 410 provides the driving force for moving the fluid. The external pump 410 draws fluid out of inlet/outlet port 408 and returns fluid through inlet/outlet port 409 via fluid line 412. Fluid lines 411 and 412 can include air bubble detector 415, conductivity sensor 416, and temperature sensor 417. Any number of additional sensors and combinations of sensors can be added. However, any additional or alternative sensors can be included. The fluid used in FIG. 3B is a concentrated solution of solutes used for peritoneal dialysis fluid. Conductivity sensor 416 can be used to ensure that the concentration of the solutes in the concentrated solution are within a predetermined range. Fluid can re-enter the peritoneal dialysis cassette through inlet/outlet port 409 and flow to inlet/outlet port 405, fluidly connected to a peritoneal dialysis fluid bag. Fluid from additional concentrate sources can also be added to the peritoneal dialysis fluid bag. Closing valve 418 and opening valve 419 will allow fluid from a peritoneal dialysis fluid source connected to inlet/outlet port 402 to be added to the peritoneal dialysis fluid bag. Closing valve 419 and opening valve 420 will allow fluid from a peritoneal dialysis fluid source connected to inlet/outlet port 403 to be added to the peritoneal dialysis fluid bag. Closing valve 420 and opening valve 421 will allow fluid from a peritoneal dialysis fluid source connected to inlet/outlet port 404 to be added to the peritoneal dialysis fluid bag. Any number of peritoneal dialysis fluid sources can be added in a similar fashion.

Once the peritoneal dialysis fluid bag is filled and mixed, the peritoneal dialysis cassette can be primed with the peritoneal dialysis fluid. FIG. 3C shows the initial steps of priming the peritoneal dialysis cassette to the drain. Closing valves 418, 426, and 427 and opening valves 422 and 425A allow the external pump 410 to draw fluid from the peritoneal dialysis fluid bag through inlet/outlet port 405 to inlet/outlet port 407 using the external pump 410 by the fluid line 411 and 412.

Once the fluid pathways that will be used for filling the patient are primed, the pinch valve switching can take place to activate the paths for patient infusion. As described, air bubble detector 415 is used to ensure the peritoneal dialysis fluid does not contain any air bubbles. Conductivity sensor 416, as well as any other composition sensors ensure that the final peritoneal dialysis fluid is within predetermined limits for each solute. A temperature sensor 417 can be used to ensure that the peritoneal dialysis fluid is heated to the proper temperature prior to infusion into the patient.

FIG. 3D illustrates the process of filling the patient with peritoneal dialysis fluid. Valves 418-421, 425A-B and 426 are closed and valves 422-424 and 427 are opened. The fluid now travels from the peritoneal dialysis bag fluidly connected to inlet/outlet port 408, through pump 410 to inlet/outlet port 409, and then to a patient line fluidly connected to inlet/outlet port 414. The patient line can be connected to a catheter for infusion of the peritoneal dialysis fluid into the peritoneal cavity of the patient. While filling the patient, the fluid can still be checked by sensors connected in fluid lines 411 and 412 to ensure that the composition of the peritoneal dialysis fluid remains correct and that no air bubbles are in the fluid. A temperature sensor 417 can be used to ensure that the peritoneal dialysis fluid remains at the proper temperature prior to infusion into the patient. A pressure sensor at position 428 and a pressure sensor at position 429 can be used to measure the fluid pressure during filling of the patient. The system can ensure that the fluid pressure is not too high during filling, and can use the fluid pressure to determine when the patient is full of peritoneal dialysis fluid.

After the peritoneal dialysis fluid has dwelled in the peritoneal cavity of the patient, the used peritoneal dialysis fluid can be drained from the patient as illustrated in FIG. 3E. Valves 418-421, 423, 425B and 427 are closed, while valves 422, 424, 426 and 425A are opened. Fluid can now travel from inlet/outlet port 414 fluidly connected to a patient line, through the peritoneal dialysis cassette to inlet/outlet port 407, fluidly connected to a drain line. By using sensors on the fluid line 411 and 412, the system can measure fluid characteristics of the used peritoneal dialysis fluid, such as the ending solute concentrations. The fluid characteristics can be used by the system or health care providers to adjust the therapy prescription as needed.

FIG. 3F illustrates the use of automatic waste fluid collection for measurement of the used peritoneal dialysate. During draining, valve 425A can be closed and valve 425B can be opened. Used peritoneal dialysis fluid can now flow through inlet/outlet port 413 rather than inlet/outlet port 407. Inlet/outlet port 413 can be connected to a sampling bag to collect a volume of used peritoneal dialysis fluid. The collected used peritoneal dialysis fluid can be analyzed later to measure any fluid characteristic of the used peritoneal dialysis fluid. The waste fluid collection can be an automatic waste fluid collection, where the control system is programmed to collect a preset volume of spent dialysis fluid at specified times, or can be manual, where a user directs the system to collect spent dialysis fluid as needed. The amount of spent dialysis fluid collected can vary depending on the needs of the system or user. In certain embodiments, volume below 1000 mL of spent peritoneal dialysis fluid can be collected. After the necessary volume of used peritoneal dialysis fluid has been collected, valve 425B can be closed and valve 425A can be opened to continue draining the used peritoneal dialysis fluid from the patient, as illustrated in FIG. 3E.

One skilled in the art will understand that various combinations and/or modifications and variations can be made in the described systems and methods depending upon the specific needs for operation. Various aspects disclosed herein may be combined in different combinations than the combinations specifically presented and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be required to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A peritoneal dialysis cassette, comprising:
a cassette housing having a plurality of fluid channels fluidly connected to a plurality of inlet/outlet ports;
wherein the plurality of inlet/outlet ports comprise at least a first inlet port fluidly connectable to an external pump and a second inlet/outlet port fluidly connectable to the external pump; and
one or more pinch valve positions in the plurality of fluid channels to selectively direct fluid from a first specified inlet/outlet port to a second specified inlet/outlet port; and
the external pump fluidly connectable to the cassette housing via the plurality of inlet/outlet ports.

2. The peritoneal dialysis cassette of claim 1, further comprising a conductivity sensor in a fluid line fluidly connecting the external pump to either the first or second inlet/outlet port.

3. The peritoneal dialysis cassette of claim 1, further comprising an air bubble detector in a fluid line fluidly connecting the external pump to either the first or second inlet/outlet port.

4. The peritoneal dialysis cassette of claim 1, further comprising at least one temperature sensor in the peritoneal dialysis cassette.

5. The peritoneal dialysis cassette of claim 1, further comprising at least one pressure sensor in the peritoneal dialysis cassette.

6. A peritoneal dialysis system, comprising:
the peritoneal dialysis cassette of claim 1;
a first peritoneal dialysis fluid source fluidly connectable to a third inlet/outlet port;
a peritoneal dialysis fluid bag fluidly connectable to a fourth inlet/outlet port;
and a control system in communication with one or more pinch valves each positioned at one of the one or more pinch valve positions; the control system programmed to control the one or more pinch valves to selectively direct fluid through the peritoneal dialysis cassette.

7. The system of claim 6, further comprising at least a second peritoneal dialysis fluid source fluidly connectable to a fifth inlet/outlet port; the control system programmed to control the one or more valves to selectively direct fluid from the first peritoneal dialysis fluid source to the peritoneal dialysis fluid bag; and then to selectively direct fluid from the second peritoneal dialysis fluid source to the peritoneal dialysis fluid bag.

8. The system of claim 6, further comprising a fifth inlet/outlet port fluidly connectable to a patient line; the control system programmed to control the one or more pinch valves to selectively direct fluid from the peritoneal dialysis fluid bag into the patient line.

9. The system of claim 6, further comprising a fifth inlet/outlet port fluidly connectable to a drain line; the control system programmed to control the one or more pinch valves to selectively direct fluid from the peritoneal dialysis fluid bag into the drain line.

10. The system of claim 8, further comprising a sixth inlet/outlet port fluidly connectable to a drain line; the control system programmed to control the one or more pinch valves to selectively direct fluid from the patient line into the drain line.

11. The system of claim 6, further comprising a fifth inlet/outlet port fluidly connected to a sampling line.

12. The system of claim 11, wherein the sampling line is fluidly connected to a sampling bag.

13. The system of claim 6, further comprising a conductivity sensor in a fluid line fluidly connecting the external pump to either the first or second inlet/outlet port.

14. The system of claim 12, further comprising a sixth inlet/outlet port fluidly connectable to a patient line; wherein the control system is programmed to control the one or more pinch valves to selectively direct fluid from the fourth inlet/outlet port to the seventh inlet/outlet port.

15. The system of claim 11, further comprising an air bubble detector in a fluid line connecting the external pump to either the first or second inlet/outlet port.

16. The system of claim 15, further comprising a sixth inlet/outlet port fluidly connectable to a patient line; wherein the control system is programmed to control the one or more pinch valves to selectively direct fluid from the fourth inlet/outlet port to the sixth inlet/outlet port.

17. A method, using the peritoneal dialysis cassette of claim 1; comprising the steps of:
pumping fluid from a third inlet/outlet port fluidly connected to a peritoneal dialysis fluid source, through the peritoneal dialysis cassette to a fourth inlet/outlet port fluidly connected to a peritoneal dialysis fluid bag.

18. The method of claim 17, further comprising the step of pumping fluid from the peritoneal dialysis fluid bag into a patient line fluidly connected to a fifth inlet/outlet port; the patient line fluidly connectable to a catheter.

19. The method of claim 18, further comprising the step of measuring at least one fluid characteristic of fluid in a fluid line fluidly connecting the external pump to either the first or second inlet/outlet port prior to pumping the fluid to the patient line.

20. The method of claim 19, wherein the fluid line fluidly connecting the external pump to either the first or second inlet/outlet port comprises at least a conductivity sensor and an air bubble detector.
